(19) United States
(12) Patent Application Publication
(10) Pub. No.: US 2025/0110076 A1
(43) Pub. Date: Apr. 3, 2025

(54) SENSOR ELEMENT AND GAS SENSOR

(71) Applicant: NGK INSULATORS, LTD., Nagoya-shi (JP)

(72) Inventors: Yusuke WATANABE, Nagoya-city (JP); Ryo HASHIKAWA, Gifu-city (JP)

(21) Appl. No.: 18/979,857

(22) Filed: Dec. 13, 2024

Related U.S. Application Data
(63) Continuation of application No. PCT/JP2023/021670, filed on Jun. 12, 2023.

(30) Foreign Application Priority Data
Jun. 17, 2022 (JP) 2022-097719

(51) Int. Cl. G01N 27/407; G01N 27/406; G01N 27/41; G01N 33/00

(52) U.S. Cl. CPC G01N 27/4076; G01N 27/4067; G01N 27/41; G01N 33/0027

(57) ABSTRACT

A sensor element includes an element body, a reference electrode, a measurement-object gas side electrode, a first conducting section that has a reference electrode terminal and a reference electrode lead, and a lead insulation layer. The reference electrode lead is configured to flow a reference gas, which serves as a reference for detecting the specific gas concentration in the measurement-object gas, to the reference electrode. A region of 50% or more, from an end on the reference electrode side, of the total area of the lead insulation layer in a planar view is configured as a dense region with a porosity of 5% or less.

### CROSS-REFERENCE TO RELATED APPLICATION

This application is a continuation application of PCT/JP2023/021670, filed on Jun. 12, 2023, which claims the benefit of priority of Japanese Patent Application No. 2022-097719, filed on Jun. 17, 2022, the entire contents of which are incorporated herein by reference.

### BACKGROUND OF THE INVENTION

### 1. Field of the Invention

The present invention relates to a sensor element and a gas sensor.

### 2. Description of the Related Art

A known gas sensor in the related art is configured to detect a specific gas concentration such as NOx in a measurement-object gas such as an exhaust gas from an automobile. PTL 1 discloses a gas sensor including a sensor element that comprises: an element body with an oxygen-ion-conductive solid electrolyte layer and a measurement-object gas flow section located inside the element body that introduces and flows the measurement-object gas; a reference electrode disposed inside the element body; and a porous reference-gas introduction layer that introduces the reference gas used as a reference for detection of the specific gas concentration in the measurement-object gas, and flows the reference gas to the reference electrode.

### CITATION LIST

### Patent Literature

PTL 1: JP 2018-173320 A

### SUMMARY OF THE INVENTION

In the sensor element described above in PTL 1, the porous reference gas introduction layer may adsorb external water (water vapor) during the non-operational period of the sensor element. In such cases, the water gradually drains out of the element body after the sensor element starts operating. However, until the water has been sufficiently drained, the oxygen concentration around the reference electrode may decrease. If the time required for the water to drain out (water drain time) is long, the time from the start of the operation of the sensor element until the potential of the reference electrode stabilizes (stabilization time) tends to be longer.

The main object of the sensor element and gas sensor according to the present invention is to shorten the stability time.

In order to achieve the above main object, the sensor element and gas sensor according to the present invention employs the following configuration.

[1] A sensor element according to the present invention is a sensor element configured to detect a specific gas concentration in a measurement-object gas, the sensor element comprising: an element body with an oxygen-ion-conductive solid electrolyte layer and a measurement-object gas flow section located inside the element body that introduces and flows the measurement-object gas; a reference electrode disposed inside the element body; a measurement-object gas side electrode disposed on a portion of the element body that is exposed to the measurement-object gas; a first conducting section including a reference electrode terminal disposed on an outside of the element body, and a reference electrode lead that is electrically continuous between the reference electrode terminal and the reference electrode; and a lead insulation layer covering a portion of the reference electrode lead that is located inside the element body, wherein the reference electrode lead is configured to flow a reference gas, which serves as a reference for detecting the specific gas concentration in the measurement-object gas, to the reference electrode, and a region of 50% or more, from an end on the reference electrode side, of the total area of the lead insulation layer in a planar view is configured as a dense region with a porosity of 5% or less.

In the sensor element according to the present invention, the reference electrode lead is configured to flow the reference gas, which serves as the reference for detecting the specific gas concentration in the measurement-object gas, to the reference electrode, and the region of 50% or more, from an end on the reference electrode side, of the total area of the lead insulation layer in the planar view is configured as the dense region with the porosity of 5% or less. Thus, as the region of 50% or more of the total area of the lead insulation layer in the planar view is configured as the dense region, the amount of water (water vapor) adsorbed by the lead insulation layer during the non-operational period of the sensor element is reduced compared to a case where less than 50% of the total area is configured as the dense region. Additionally, as the region that includes at least the end on the reference electrode side of the lead insulation layer is configured as the dense region, even if the lead insulation layer adsorbs water during the non-operational period of the sensor element, the water drains more easily from the element body after the sensor element starts operating, compared to a case where a porous region with a porosity exceeding 5% is provided on the reference electrode side relative to the dense region in the lead insulation layer. Consequently, the time from the start of the operation of the sensor element until the water drains out of the element body (water drain time) is shortened, which in turn shortens the time from the start of the operation of the sensor element until the potential of the reference electrode stabilizes (stabilization time). The inventors have confirmed this through experiments and other methods.

[2] In the sensor element above (the sensor element according to [1] above), the region of 80% or more, from the end on the reference electrode side, of the total area in the planar view may be configured as the dense region. Such a configuration further reduces the amount of water (water vapor) adsorbed by the lead insulation layer during the non-operational period of the sensor element.

[3] In the sensor element above (the sensor element according to [1] or [2] above), the limiting current when oxygen is pumped from the surroundings of the reference electrode to the surroundings of the measurement-object gas side electrode may be 0.3 μA or more. Here, the limiting current has a positive correlation with the inverse of the diffusion resistance of the reference electrode lead. A higher limiting current indicates a lower diffusion resistance of the reference electrode lead. Therefore, a limiting current of 0.3 μA or more indicates that the diffusion resistance of the reference electrode lead is at or below a predetermined value (i.e., not excessively high). Such a configuration facilitates the maintenance of the oxygen concentration around the reference electrode at an appropriate level compared to a case where the diffusion resistance of the reference electrode lead is excessively high, thereby reducing the decrease in detection accuracy of the specific gas concentration. The inventors have confirmed this through experiments and other methods. In this case, the limiting current may also be 10 μA or less.

[4] In the sensor element above (the sensor element according to [3] above), the limiting current may be 0.5 μA or more. Such a configuration further reduces the decrease in the detection accuracy of the specific gas concentration.

[5] In the sensor element above (the sensor element according to any one of [1] to [4] above), the porosity of a portion of the reference electrode lead that is located inside the element body is more than 5% and 40% or less. As the porosity is more than 58, the reference gas is more likely to flow to the reference electrode. Additionally, as the porosity is 40% or less, the portion of the reference electrode lead that is located inside the element body is less likely to become disconnected.

[6] In the sensor element above (the sensor element according to any one of [1] to [5] above), a cross-sectional area and/or a porosity of a portion of the reference electrode lead that is located inside the element body may be greater on the reference electrode side than on the reference electrode terminal side. Such a configuration increases the likelihood of oxygen accumulating around the reference electrode, thereby facilitating the maintenance of the oxygen concentration around the reference electrode at the appropriate level.

[7] In the sensor element above (the sensor element according to any one of [1] to [6] above), the sensor element further may include: a heater disposed inside the element body and heating the element body; a second conducting section including a heater terminal disposed on the outside of the element body, and a heater lead that is electrically continuous between the heater terminal and the heater; and a heater insulation layer covering both the heater and a portion of the heater lead that is located inside the element body. The lead insulation layer and the heater insulation layer may be independently provided such that they do not connect with each other.

[8] In the sensor element above (the sensor element according to any one of [1] to [6] above), the sensor element further may include: a heater disposed inside the element body and heating the element body; a second conducting section including a heater terminal disposed on the outside of the element body, and a heater lead that is electrically continuous between the heater terminal and the heater; and a heater insulation layer covering both the heater and a portion of the heater lead that is located inside the element body. The element body may include a communication hole that connects the heater insulation layer and the lead insulation layer, and a porosity of the heater insulation layer may be 5% or less. Such a configuration reduces the amount of water (water vapor) adsorbed by the heater insulation layer via the lead insulation layer and the communication hole during the non-operational period of the sensor element.

[9] A gas sensor according to the present invention includes the sensor element according to any one of [1] to [7] above. Therefore, the gas sensor provides the same advantageous effects as those achieved by the sensor element according to the present invention, for example, an advantageous effect of shortening the stabilization time.

### BRIEF DESCRIPTION OF THE DRAWINGS

FIG. 1 is a vertical cross-sectional view of a gas sensor 100.

FIG. 2 is a schematic cross-sectional view of a sensor element 101, illustrating an exemplary configuration thereof.

FIG. 3 is a block diagram illustrating an electrical connection relationship between a control device 95 and relevant elements including cells.

FIG. 4 is a partially enlarged cross-sectional view of an area around a reference electrode 42 and others.

FIG. 5 is a cross-sectional view taken along A-A in FIG. 4.

FIG. 6 is a perspective view of a rear end of an element body 102.

FIG. 7 is a cross-sectional view of a part of a sensor element 201 according to a modification.

FIG. 8 is a cross-sectional view of a part of a sensor element 301 according to another modification.

FIG. 9 is a cross-sectional view of a part of a sensor element 401 according to yet another modification.

FIG. 10 is a cross-sectional view of a part of a sensor element 501 according to yet another modification.

FIG. 11 is a cross-sectional view of a part of a sensor element 601 according to yet another modification.

FIG. 12 is a cross-sectional view taken along B-B in FIG. 11.

FIG. 13 is a schematic cross-sectional view of a sensor element 701 according to yet another modification.

### DETAILED DESCRIPTION OF THE INVENTION

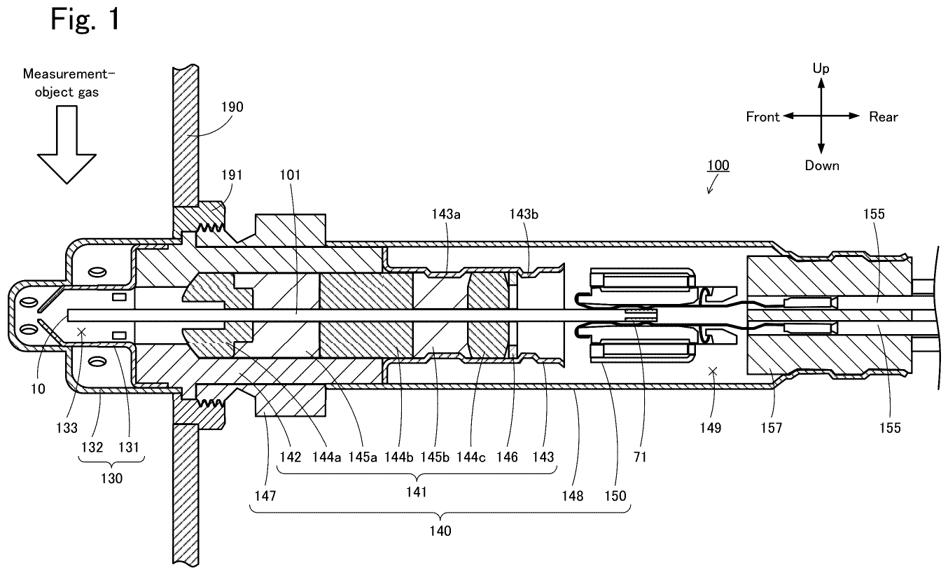

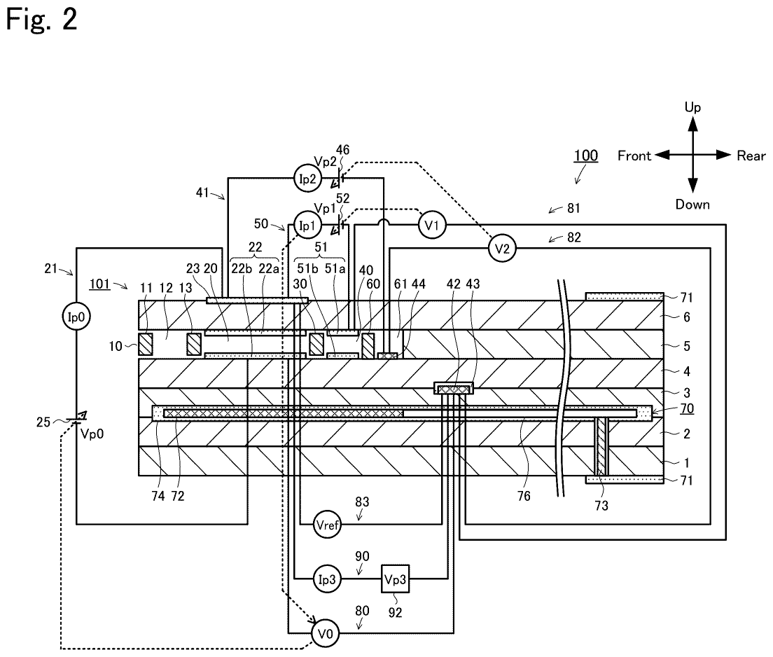

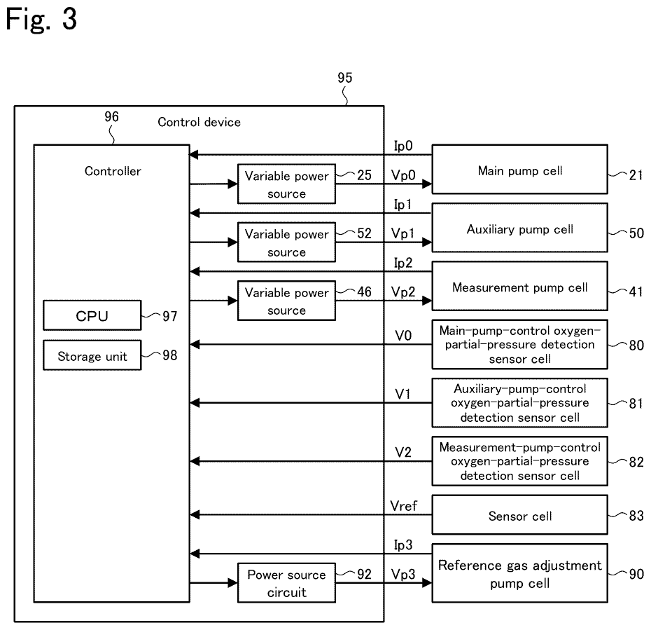

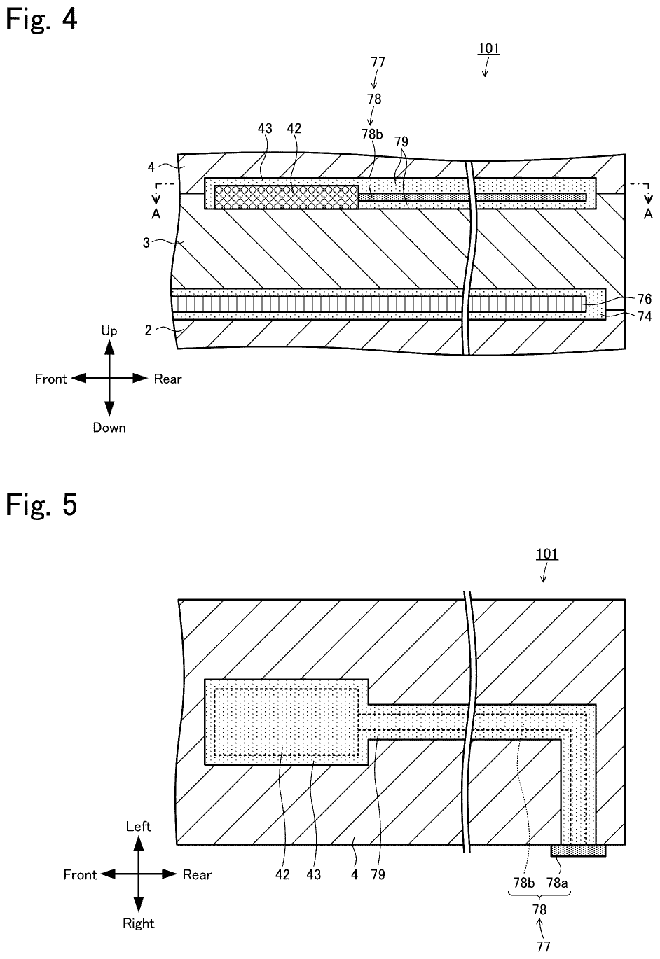

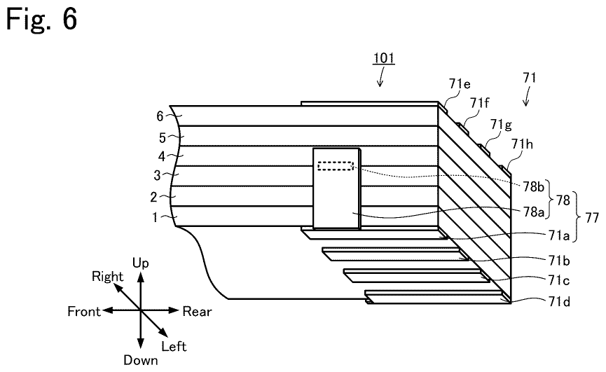

Embodiments of the present invention will now be described with reference to the drawings. FIG. 1 is a vertical cross-sectional view of a gas sensor 100 according to an embodiment of the present invention. FIG. 2 is a schematic cross-sectional view of a sensor element 101 included in the gas sensor 100, illustrating an exemplary configuration thereof. FIG. 3 is a block diagram illustrating an electrical connection relationship between a control device 95 and relevant elements including cells. FIG. 4 is a partially enlarged cross-sectional view of an area around a reference electrode 42, a reference electrode lead 78 and a lead 76 of the sensor element 101. FIG. 5 is a cross-sectional view taken along A-A in FIG. 4. FIG. 6 is a perspective view of a rear end of an element body 102. The sensor element 101 has an elongate rectangular parallelepiped shape. The longitudinal direction of the sensor element 101 (the left-right direction in FIG. 2) is defined as the front-rear direction. The thickness direction of the sensor element 101 (the up-down direction in FIG. 2) is defined as the up-down direction. The width direction of the sensor element 101 (the direction perpendicular to the front-rear direction and to the up-down direction) is defined as the left-right direction.

As illustrated in FIG. 1, the gas sensor 100 includes the sensor element 101, a protection cover 130 protecting a part of the sensor element 101 that is at the front end, and a sensor assembly 140 including a connector 150 electrically continuous with the sensor element 101. As illustrated in the drawing, the gas sensor 100 is attached to a pipe 190, such as an exhaust pipe of an automobile. The gas sensor 100 is used to measure specific gas concentration, such as NOx or O₂, contained in exhaust gas as the measurement-object gas. In the present embodiment, the gas sensor 100 is configured to measure NOx concentration as the specific gas concentration.

The protection cover 130 includes a bottomed cylindrical inner protection cover 131 covering the front end of the sensor element 101, and a bottomed cylindrical outer protection cover 132 covering the inner protection cover 131. The inner protection cover 131 and the outer protection cover 132 each have a plurality of holes for allowing the measurement-object gas to flow into the protection cover 130. The space enclosed by the inner protection cover 131 serves as a sensor element chamber 133. The front end of the sensor element 101 is positioned in the sensor element chamber 133.

The sensor assembly 140 includes an element sealing unit 141 that seals and secures the sensor element 101, a bolt 147 and an outer cylinder 148 that are attached to the element sealing unit 141, and the connector 150 that is in contact with and electrically connected to connector electrodes 71 disposed on surfaces (upper and lower surfaces) of the sensor element 101 at a rear end.

The element sealing unit 141 includes a cylindrical main metal fitting 142, and a cylindrical inner cylinder 143 welded coaxially to the main metal fitting 142. The element sealing unit 141 further includes ceramic supporters 144a to 144c, compacts 145a and 145b, and a metal ring 146 that are sealed in a through-hole provided on the inner side of the main metal fitting 142 and the inner cylinder 143. The sensor element 101 is located on the center axis of the element sealing unit 141 and extends through the element sealing unit 141 in the front-rear direction. The inner cylinder 143 has a diameter reduction section 143a for pressing the compact 145b toward the center axis of the inner cylinder 143, and a diameter reduction section 143b for pressing the ceramic supporters 144a to 144c and the compacts 145a and 145b frontward via the metal ring 146. The pressing forces generated by the diameter reduction sections 143a and 143b compress the compacts 145a and 145b between the sensor element 101 and the combination of the main metal fitting 142 and the inner cylinder 143. Thus, the compacts 145a and 145b seal between the sensor element chamber 133 in the protection cover 130 and a space 149 in the outer cylinder 148, and also secure the sensor element 101.

The bolt 147 is secured coaxially to the main metal fitting 142 and has a male threaded section around the outer peripheral surface thereof. The male threaded section of the bolt 147 is screwed into a securing member 191 welded to the pipe 190 and having a female threaded section around the inner peripheral surface thereof. Thus, the gas sensor 100 is secured to the pipe 190, with a part of the gas sensor 100 inclusive of the front end of the sensor element 101 and the protection cover 130 protruding into the pipe 190.

The outer cylinder 148 encloses the inner cylinder 143, the sensor element 101, and the connector 150. A plurality of lead wires 155 are connected to the connector 150 and are routed to the outside from the rear end of the outer cylinder 148. The lead wires 155 are electrically continuous with electrodes (to be described below) of the sensor element 101 via the connector 150. The gap between the outer cylinder 148 and the lead wires 155 is sealed by a rubber stopper 157. The space 149 in the outer cylinder 148 is filled with a reference gas. The rear end of the sensor element 101 is positioned in the space 149.

As illustrated in FIG. 2, the sensor element 101 is an element including a layered body obtained by stacking six layers in the following order from the lower side in the drawing: a first substrate layer 1, a second substrate layer 2, a third substrate layer 3, a first solid electrolyte layer 4, a spacer layer 5, and a second solid electrolyte layer 6, which are each an oxygen-ion-conductive solid electrolyte layer composed of zirconia (ZrO₂) or the like. The solid electrolyte forming each of these six layers is dense and hermetic. The sensor element 101 is manufactured by, for example, performing predetermined processing and printing of circuit patterns on ceramic green sheets that are to become the individual layers, subsequently stacking the sheets, and then combining the sheets by performing calcination.

At an end (the left side in FIG. 2) of the sensor element 101 and between the lower surface of the second solid electrolyte layer 6 and the upper surface of the first solid electrolyte layer 4 are provided a gas inlet 10, a first diffusion control section 11, a buffer space 12, a second diffusion control section 13, a first internal cavity 20, a third diffusion control section 30, a second internal cavity 40, a fourth diffusion control section 60, and a third internal cavity 61 that are next to one another and connect with one another in that order.

The gas inlet 10, the buffer space 12, the first internal cavity 20, the second internal cavity 40, and the third internal cavity 61 are spaces provided inside the sensor element 101 by hollowing out the spacer layer 5, and each have an upper side defined by the lower surface of the second solid electrolyte layer 6, a lower side defined by the upper surface of the first solid electrolyte layer 4, and lateral sides defined by the side surfaces of the spacer layer 5.

The first diffusion control section 11, the second diffusion control section 13, and the third diffusion control section 30 are each provided as two horizontally elongate slits (i.e., openings that are elongate in the direction perpendicular to the plane of the drawing). The fourth diffusion control section 60 is provided as a single horizontally elongate slit (i.e., an opening that is elongate in the direction perpendicular to the plane of the drawing) providing a gap with respect to the lower surface of the second solid electrolyte layer 6. A section extending from the gas inlet 10 to the third internal cavity 61 inclusive is also referred to as a measurement-object gas flow section.

The reference electrode 42 is disposed between the upper face of the third substrate layer 3 and the lower face of the first solid electrolyte layer 4. This reference electrode 42 is disposed directly on the upper face of the third substrate layer 3, and the upper, front, rear, left and right faces of the reference electrode 42 are covered by the reference electrode insulation layer 43. Additionally, the reference electrode 42 is formed as a porous cermet electrode (for example, a cermet electrode of Pt and Zro₂). As will be described below, the reference electrode 42 receives a reference gas that is used as a reference for detecting the NOx concentration, flowing from outside the sensor element 101 (here, the space 149 in FIG. 1) via the reference electrode lead 78 (see FIGS. 4 and 5) connected to the reference electrode 42. The reference gas is a gas with a predetermined oxygen concentration, and in the present embodiment, it is either an atmosphere or a gas with the same oxygen concentration as the atmosphere (for example, a gas containing oxygen with nitrogen as the base gas). In addition, as will be described below, the reference electrode 42 is used to measure the oxygen concentration (oxygen partial pressure) in the first internal cavity 20, the second internal cavity 40, and the third internal cavity 61. The reference electrode insulation layer 43 is provided for the purpose of obtaining electrical insulation between the first solid electrolyte layer 4 and the reference electrode 42. Details regarding the reference electrode insulation layer 43 will be described below.

In the measurement-object gas flow section, the gas inlet 10 is open to an external space, so that the measurement-object gas is to be taken into the sensor element 101 from the external space through the gas inlet 10. The first diffusion control section 11 is provided for applying a predetermined diffusion resistance to the measurement-object gas taken in through the gas inlet 10. The buffer space 12 is provided for guiding the measurement-object gas introduced through the first diffusion control section 11 to the second diffusion control section 13. The second diffusion control section 13 is provided for applying a predetermined diffusion resistance to the measurement-object gas to be introduced to the first internal cavity 20 from the buffer space 12. When the measurement-object gas is introduced from outside the sensor element 101 to the first internal cavity 20, the measurement-object gas quickly taken into the sensor element 101 through the gas inlet 10 because of pressure fluctuation of the measurement-object gas (i.e., pulsation of exhaust pressure, if the measurement-object gas is exhaust gas of an automobile) in the external space is not directly introduced to the first internal cavity 20 but is introduced to the first internal cavity 20 after the pressure fluctuation of the measurement-object gas is negated by traveling through the first diffusion control section 11, the buffer space 12, and the second diffusion control section 13. Accordingly, the pressure fluctuation of the measurement-object gas to be introduced to the first internal cavity 20 becomes substantially negligible. The first internal cavity 20 is provided as a space for adjusting the oxygen partial pressure in the measurement-object gas introduced thereto via the second diffusion control section 13. The oxygen partial pressure is adjusted by actuating a main pump cell 21.

The main pump cell 21 is an electrochemical pump cell constituted of an inner pump electrode 22 including a ceiling electrode portion 22a disposed on the lower surface of the second solid electrolyte layer 6 over substantially the entirety of an area that faces the first internal cavity 20; an outer pump electrode 23 disposed on the upper surface of the second solid electrolyte layer 6 over an area that corresponds to the ceiling electrode portion 22a in such a manner as to be exposed to the outside space (the sensor element chamber 133 in FIG. 1); and the second solid electrolyte layer 6 that is interposed between the electrodes 22 and 23.

The inner pump electrode 22 is provided astride the upper and lower solid electrolyte layers (i.e., the second solid electrolyte layer 6 and the first solid electrolyte layer 4) that define the first internal cavity 20, and the spacer layer 5 that provides sidewalls of the first internal cavity 20. Specifically, the lower surface of the second solid electrolyte layer 6 that provides the ceiling surface of the first internal cavity 20 is provided with the ceiling electrode portion 22a, the upper surface of the first solid electrolyte layer 4 that provides the bottom surface of the first internal cavity 20 is directly provided with a bottom electrode portion 22b, and the ceiling electrode portion 22a and the bottom electrode portion 22b are connected to each other by lateral electrode portions (not illustrated) provided on respective sidewalls (inner surfaces) of the spacer layer 5 that form opposite sidewalls of the first internal cavity 20. Thus, the inner pump electrode 22 has a tunnel-like structure in a region where the lateral electrode portions are provided.

The inner pump electrode 22 and the outer pump electrode 23 each are formed as a porous cermet electrode (for example, a cermet electrode made of Pt and ZrO₂, having an Au content of 1 percent). The inner pump electrode 22 that contacts with a measurement-object gas is formed by using a material of which a reduction ability for NOx components in the measurement-object gas is lowered.

In the main pump cell 21, a desired pump voltage Vp0 is applied between the inner pump electrode 22 and the outer pump electrode 23, whereby a pump current Ip0 is caused to flow in the positive direction or the negative direction between the inner pump electrode 22 and the outer pump electrode 23. Thus, the oxygen in the first internal cavity 20 is pumped out to the external space, or the oxygen in the external space is pumped into the first internal cavity 20.

Furthermore, in order to detect the oxygen concentration (oxygen partial pressure) in the atmosphere within the first internal cavity 20, the inner pump electrode 22, the second solid electrolyte layer 6, the spacer layer 5, the first solid electrolyte layer 4, the third substrate layer 3, and the reference electrode 42 constitute an electrochemical sensor cell, that is, a main-pump-control oxygen-partial-pressure detection sensor cell 80.

The oxygen concentration (oxygen partial pressure) in the first internal cavity 20 is to be determined by measuring an electromotive force (voltage V0) in the main-pump-control oxygen-partial-pressure detection sensor cell 80. Furthermore, feedback control is performed on the pump voltage Vp0 of a variable power source 25 such that the voltage V0 reaches a fixed value (target value), whereby the pump current Ip0 is controlled. Thus, the oxygen concentration in the first internal cavity 20 is maintained at a predetermined fixed value.

The third diffusion control section 30 is provided for applying a predetermined diffusion resistance to the measurement-object gas having an oxygen concentration (oxygen partial pressure) controlled in the first internal cavity 20 under the operation of the main pump cell 21, and then guiding the measurement-object gas to the second internal cavity 40.

The second internal cavity 40 is provided as a space for an auxiliary pump cell 50 to further adjust the oxygen partial pressure in the measurement-object gas having an oxygen concentration (oxygen partial pressure) preliminarily adjusted in the first internal cavity 20 and introduced to the second internal cavity 40 via the third diffusion control section 30. Thus, the oxygen concentration in the second internal cavity 40 is maintained at a fixed level with high accuracy, which enables the gas sensor 100 to achieve highly accurate NOx concentration measurement.

The auxiliary pump cell 50 is an auxiliary electrochemical pump cell constituted of an auxiliary pump electrode 51 including a ceiling electrode portion 51a disposed on the lower surface of the second solid electrolyte layer 6 over substantially the entirety of an area that faces the second internal cavity 40; the outer pump electrode 23 (but not limited to the outer pump electrode 23 and may possibly be an appropriate electrode disposed on outside the sensor element 101); and the second solid electrolyte layer 6.

The auxiliary pump electrode 51 is disposed in the second internal cavity 40 and has a tunnel-like structure similar to the inner pump electrode 22 disposed in the first internal cavity 20. Specifically, the second solid electrolyte layer 6 that provides the ceiling surface of the second internal cavity 40 is provided with the ceiling electrode portion 51a, the upper surface of first solid electrolyte layer 4 that provides the bottom surface for the second internal cavity 40 is directly provided with a bottom electrode portion 51b, and the ceiling electrode portion 51a and the bottom electrode portion 51b are connected to each other by lateral electrode portions (not illustrated) provided on opposite wall surfaces of the spacer layer 5 that provide respective sidewalls of the second internal cavity 40. Thus, the auxiliary pump electrode 51 has a tunnel-like structure. Note that the auxiliary pump electrode 51 is also formed by using a material of which the reduction ability for the NOx components in the measurement-object gas is lowered, as with the inner pump electrode 22.

In the auxiliary pump cell 50, a desired voltage Vp1 is applied between the auxiliary pump electrode 51 and the outer pump electrode 23 so that the oxygen in the atmosphere within the second internal cavity 40 can be pumped out to the external space or the oxygen can be pumped into the second internal cavity 40 from the external space.

Furthermore, in order to control the oxygen partial pressure in the atmosphere within the second internal cavity 40, the auxiliary pump electrode 51, the reference electrode 42, the second solid electrolyte layer 6, the spacer layer 5, the first solid electrolyte layer 4, and the third substrate layer 3 constitute an electrochemical sensor cell, that is, an auxiliary-pump-control oxygen-partial-pressure detection sensor cell 81.

A variable power source 52 whose voltage is controlled based on an electromotive force (voltage V1) detected by the auxiliary-pump-control oxygen-partial-pressure detection sensor cell 81 causes the auxiliary pump cell 50 to perform pumping. Thus, the oxygen partial pressure in the atmosphere in the second internal cavity 40 is controlled to be a low partial pressure that has substantially no effect on NOx measurement.

Meanwhile, a pump current Ip1 generated by the auxiliary pump cell 50 is used in controlling the electromotive force of the main-pump-control oxygen-partial-pressure detection sensor cell 80. Specifically, the pump current Ip1 is input as a control signal to the main-pump-control oxygen-partial-pressure detection sensor cell 80, and the above target value for the voltage V0 of the main-pump-control oxygen-partial-pressure detection sensor cell 80 is controlled, whereby the gradient of the oxygen partial pressure in the measurement-object gas to be introduced to the second internal cavity 40 from the third diffusion control section 30 is controlled to be constantly fixed. To use the gas sensor 100 as a NOx sensor, the oxygen concentration in the second internal cavity 40 is maintained at a fixed value of about 0.001 ppm under the cooperation of the main pump cell 21 and the auxiliary pump cell 50.

The fourth diffusion control section 60 is provided for applying a predetermined diffusion resistance to the measurement-object gas having an oxygen concentration (oxygen partial pressure) controlled in the second internal cavity 40 under the operation of the auxiliary pump cell 50, and then guiding the measurement-object gas to the third internal cavity 61. The fourth diffusion control section 60 has a role of limiting the amount of NOx flowing into the third internal cavity 61.

The third internal cavity 61 is provided as a space where a process for measuring the concentration of nitrogen oxide (NOx) in the measurement-object gas is performed on the measurement-object gas having an oxygen concentration (oxygen partial pressure) preliminarily adjusted in the second internal cavity 40 and introduced to the third internal cavity 61 via the fourth diffusion control section 60. The NOx concentration is measured mainly in the third internal cavity 61 under the operation of a measurement pump cell 41.

The measurement pump cell 41 is configured to measure the NOx concentration in the measurement-object gas in the third internal cavity 61. The measurement pump cell 41 is an electrochemical pump cell constituted of the measurement electrode 44 directly disposed on the upper surface of the first solid electrolyte layer 4 in an area that faces the third internal cavity 61; and the outer pump electrode 23, the second solid electrolyte layer 6, the spacer layer 5, and the first solid electrolyte layer 4. The measurement electrode 44 is a porous cermet electrode (for example, a cermet electrode made of Pt and ZrO₂) that is formed using a material with a higher reduction ability for NOx components in the measurement-object gas than that of the inner pump electrode 22. The measurement electrode 44 also functions as a NOx reduction catalyst that reduces the NOx existing in the atmosphere in the third internal cavity 61.

In the measurement pump cell 41, oxygen resulting from the decomposition of the nitrogen oxide in the atmosphere surrounding the measurement electrode 44 is pumped out, and the amount of oxygen thus produced is detected as a pump current Ip2.

Furthermore, in order to detect the oxygen partial pressure around the measurement electrode 44, the first solid electrolyte layer 4, the third substrate layer 3, the measurement electrode 44, and the reference electrode 42 constitute an electrochemical sensor cell, that is, a measurement-pump-control oxygen-partial-pressure detection sensor cell 82. A variable power source 46 is controlled based on an electromotive force (voltage V2) detected by the measurement-pump-control oxygen-partial-pressure detection sensor cell 82.

The measurement-object gas introduced to the second internal cavity 40 reaches the measurement electrode 44 in the third internal cavity 61 via the fourth diffusion control section 60 under a condition where the oxygen partial pressure is controlled. The nitrogen oxide in the measurement-object gas surrounding the measurement electrode 44 is reduced (2NO→N₂+O₂) to produce oxygen. Then, the oxygen thus produced undergoes pumping by the measurement pump cell 41. During the pumping of the oxygen, a voltage Vp2 of the variable power source 46 is controlled such that the voltage V2 detected by the measurement-pump-control oxygen-partial-pressure detection sensor cell 82 reaches a fixed value (target value). Because the amount of oxygen produced around the measurement electrode 44 is proportional to the concentration of the nitrogen oxide in the measurement-object gas, the nitrogen oxide concentration in the measurement-object gas is calculated by using the pump current Ip2 of the measurement pump cell 41.

Furthermore, the second solid electrolyte layer 6, the spacer layer 5, the first solid electrolyte layer 4, the third substrate layer 3, the outer pump electrode 23, and the reference electrode 42 constitute an electrochemical sensor cell 83. Using an electromotive force (voltage Vref) generated by the sensor cell 83, the oxygen partial pressure in the measurement-object gas outside the sensor element 101 is detectable.

Furthermore, the second solid electrolyte layer 6, the spacer layer 5, the first solid electrolyte layer 4, the third substrate layer 3, the outer pump electrode 23, and the reference electrode 42 constitute an electrochemical reference gas adjustment pump cell 90. The reference gas adjustment pump cell 90 performs oxygen pumping by allowing the pump current Ip3 to flow due to the control voltage Vp3 applied by the power source circuit 92 connected between the outer pump electrode 23 and the reference electrode 42. Thus, the reference gas adjustment pump cell 90 can either pump in the oxygen from the space surrounding the outer pump electrode 23 (the sensor element chamber 133 in FIG. 1) to the surroundings of the reference electrode 42, or pump out the oxygen from the surroundings of the reference electrode 42 to the space surrounding the outer pump electrode 23.

In the gas sensor 100 having the above configuration, the measurement pump cell 41 receives the measurement-object gas whose oxygen partial pressure is constantly maintained at a fixed low value (i.e., a value that substantially has no effect on NOx measurement) as a result of actuation of the main pump cell 21 and the auxiliary pump cell 50. Thus, the NOx concentration in the measurement-object gas can be ascertained based on the pump current Ip2 flowing as a result of oxygen produced by NOx reduction being pumped out by the measurement pump cell 41 substantially in proportion to the NOx concentration in the measurement-object gas.

Furthermore, in order to enhance the oxygen ion conductivity of the solid electrolyte, the sensor element 101 includes the heater section 70 that serves the role of temperature adjustment to keep the sensor element 101 warm by heating it. The heater section 70 includes a heater 72, a heater insulation layer 74, and a lead 76.

The heater 72 is an electrical resistor interposed between the second substrate layer 2 and the third substrate layer 3 from above and below. The heater 72 generates heat when supplied with electricity from the outside, thereby heating and maintaining the temperature of the solid electrolyte that forms the sensor element 101. Furthermore, the heater 72 is embedded in such a manner as to overlap the entire region from the first internal cavity 20 to the third internal cavity 61, and is capable of adjusting the entirety of the sensor element 101 to a temperature at which the solid electrolyte is activated.

The heater insulation layer 74 is provided for the purpose of obtaining electrical insulation between the second substrate layer 2 and both the heater 72 and the lead 76, and electrical insulation between the third substrate layer 3 and both the heater 72 and the lead 76. Details regarding the heater insulation layer 74 will be described below.

The lead 76 is formed in a manner as with the heater 72, interposed between the second substrate layer 2 and the third substrate layer 3 from above and below. The lead 76 disposed in a shape that extends from the rear end of the heater 72 toward the back, positioned slightly forward of the rear end of the sensor element 101. The rear end portion of the lead 76 is connected to the connector electrode 71 via a through-hole 73.

As illustrated in FIG. 3, the control device 95 includes the above-described variable power sources 25, 46, and 52, the above-described power source circuit 92, and a controller 96. The controller 96 is a microprocessor including a CPU 97, RAM not illustrated, a storage unit 98, and so forth. The storage unit 98 is a nonvolatile memory, such as ROM, that serves as a device for storing various types of data. The controller 96 is configured to receive the voltage V0 to V2 and Vref of each of the sensor cells 80 to 83. The controller 96 is also configured to receive the pump current Ip0 to Ip2 and Ip3 flowing through each of the pump cells 21, 50, 41, and 90. The controller 96 is configured to output control signals to the variable power sources 25, 46, and 52 and the power source circuit 92 and thus control the voltages Vp0 to Vp3 that are to be output by the variable power sources 25, 46, and 52 and the power source circuit 92, thereby controlling each of the pump cells 21, 41, 50, and 90. The controller 96 is configured to output a control signal to the heater power source which is not illustrated, and thus control the electric power that is to be supplied from the power source to the heater 72, thereby controlling the temperature of the sensor element 101. The storage unit 98 stores target values V0*, V1*, V2*, Ip1* and the like, to be described below.

The controller 96 performs feedback control on the pump voltage Vp0 of the variable power source 25 such that the voltage V0 reaches the target value V0* (i.e., such that the oxygen concentration in the first internal cavity 20 reaches a target concentration).

The controller 96 performs feedback control on the voltage Vp1 of the variable power source 52 such that the voltage V1 reaches a fixed value (referred to as the “target value V1*”) (i.e., such that the oxygen concentration in the second internal cavity 40 reaches a predetermined low oxygen concentration that has substantially no effect on NOx measurement). In conjunction with this, the controller 96 sets (i.e., performs feedback control) the target value V0* for the voltage V0 based on the pump current Ip1 such that the pump current Ip1 flowing at the voltage Vp1 reaches a fixed value (referred to as the “target value Ip1*”). Thus, the gradient of the oxygen partial pressure in the measurement-object gas to be introduced from the third diffusion control section 30 to the second internal cavity 40 is constantly fixed. Additionally, the oxygen partial pressure in the atmosphere of the second internal cavity 40 is controlled to a low level that has no substantial effect on NOx measurement. The target value V0* is set to a value at which the oxygen concentration in the first internal cavity 20 is higher than 0% and is a low concentration.

The controller 96 performs feedback control on the voltage Vp2 of the variable power source 46 such that the voltage V2 reaches a fixed value (referred to as the “target value V2*”) (i.e., such that the oxygen concentration in the third internal cavity 61 reaches a predetermined low concentration). Thus, oxygen is pumped out from the third internal cavity 61 such that the oxygen generated by the reduction of the specific gas (in this case, NOx) in the third internal cavity 61 is effectively reduced to nearly zero. Then, the controller 96 acquires the pump current Ip2 as a value detected in correspondence with the oxygen produced in the third internal cavity 61 from NOx, and calculates the NOx concentration in the measurement-object gas based on the pump current Ip2. The target value V2* is predetermined to be a value such that the pump current Ip2 flowing at the feedback-controlled voltage Vp2 reaches a limit current. The storage unit 98 stores, for example, a relational expression (for example, a linear function expression) or a map that represents the correspondence relationship between the pump current Ip2 and the NOx concentration. Such a relational expression or map can be preliminarily obtained through experiments. The controller 96 detects the NOx concentration in the measurement-object gas based on the acquired pump current Ip2 and the correspondence relationship stored in the storage unit 98.

The controller 96 controls the power source circuit 92 to apply the voltage Vp3 to the reference gas adjustment pump cell 90, thereby allowing the pump current Ip3 to flow. Thus, the controller 96 is able to pump oxygen from the surroundings of the outer pump electrode 23 to the surroundings of the reference electrode 42, and pump out the oxygen from the surroundings of the reference electrode 42 to the surroundings of the outer pump electrode 23. In the present embodiment, the voltage Vp3 is set to a direct current voltage such that the pump current Ip3 reaches a predetermined value (a constant direct current). Consequently, with the flow of the pump current Ip3, the reference gas adjustment pump cell 90 pumps a constant amount of oxygen from the surroundings of the outer pump electrode 23 to the surroundings of the reference electrode 42.

The connector electrodes 71 are each disposed at a rear end portion of the sensor element 101. As illustrated in FIG. 6, the connector electrodes 71 include connector electrodes 71a to 71d disposed at the rear end of the lower face of the sensor element 101, and connector electrodes 71e to 71h disposed at the rear end of the upper face of the sensor element 101. The connector electrodes 71 each function as a terminal that allows the sensor element 101 and the outside to be electrically continuous with each other. The connector electrodes 71e to 71f are connected one-to-one with the inner pump electrode 22, outer pump electrode 23, measurement electrode 44, and auxiliary pump electrode 51 of the sensor element 101. The connector electrode 71a is connected to the reference electrode 42 via the reference electrode lead 78, which will be described below. The connector electrode 71b is connected to the heater 72 via the through-hole 73 and the lead 76 (see FIG. 2). The connector electrodes 71c and 71d are also connected to the heater 72 via through-holes and leads that are not illustrated. The variable power sources 25, 46, and 52, and the power source circuit 92 illustrated in FIG. 2, are actually connected to each electrode (inner pump electrode 22, outer pump electrode 23, reference electrode 42, measurement electrode 44, and auxiliary pump electrode 51) via the connector electrodes 71 and connector 150 and lead 155 illustrated in FIG. 1. Power supply to the heater section 70 from the external power source is also conducted via the connector electrodes 71 and connector 150 and lead 155.

Here, the configuration around the reference electrode 42, the reference electrode lead 78, and the lead 76 will be described in detail with FIGS. 2, 4 to 6. The reference electrode 42 is connected to a conducting section 77. The conducting section 77 includes the above described connector electrode 71a and the reference electrode lead 78 that is electrically continuous between the connector electrode 71a and the reference electrode 42. The reference electrode lead 78 includes a peripheral-face lead 78a and an internal lead 78b.

The peripheral-face lead 78a is disposed on the right face of the rear end portion of the sensor element 101, connecting the connector electrode 71a and the internal lead 78b (conducting them). The internal lead 78b is disposed inside the sensor element 101, connecting the reference electrode 42 and the peripheral-face lead 78a. Specifically, the internal lead 78b is disposed between the third substrate layer 3 and the first solid electrolyte layer 4, extending backward from the reference electrode 42, bending to the right at the rear end portion of the sensor element 101, and continuing to the right face of the rear end portion of it (see FIGS. 5 and 6). The end portion of the internal lead 78b opposite the reference electrode 42 is connected to the peripheral-face lead 78a.

The conducting section 77 is a conductor mainly composed of a noble metal such as platinum (Pt) or a high-melting-point metal such as tungsten (W) or molybdenum (Mo). The conducting section 77 is preferably a cermet conductor containing a noble metal or a high-melting-point metal and zirconia that is the same as the main component of the third substrate layer 3. The reference electrode lead 78 (peripheral-face lead 78a and internal lead 78b) has a porous with a porosity exceeding 5%. Thus, the reference electrode lead 78, as described above, allows the reference gas to flow from the outside of the sensor element 101 (the space 149 illustrated in FIG. 1) to the reference electrode 42. The porosity of the reference electrode lead 78 is preferably 10% or more. Additionally, the porosity of the reference electrode lead 78, particularly the internal lead 78b, is preferably 40% or less. Furthermore, both the peripheral-face lead 78a and the internal lead 78b should be porous, and the porosity of the peripheral-face lead 78a and the porosity of the internal lead 78b may be the same or different. The reference electrode lead 78 is designed such that the limiting current when oxygen is pumped from the surroundings of the reference electrode 42 to the surroundings of the outer pump electrode 23 is 0.3 μA or more. The limiting current is preferably 0.5 μA or more. The limiting current has a positive correlation with the inverse of the diffusion resistance of the reference electrode lead 78, particularly the internal lead 78b. A higher limiting current indicates a lower diffusion resistance of the reference electrode lead 78. Therefore, a limiting current of 0.3 μA or more indicates that the diffusion resistance of the reference electrode lead 78 is at or below a predetermined value (i.e., not excessively high). The diffusion resistance of the internal lead 78b may be 3060×10³ cm⁻¹ or less, and is preferably 2000×10³ cm⁻¹ or less. The diffusion resistance of the internal lead 78b is calculated by dividing the length in the extending direction of the internal lead 78b by the product of its width, thickness, and porosity. The limiting current may also be 10 μA or less, which indicates that the diffusion resistance of the internal lead 78b is above a second predetermined value that is less than the first predetermined value (i.e., not excessively low).

The upper, front, back, left, and right faces of the reference electrode 42 are covered by the reference electrode insulation layer 43, as described above. Additionally, the outer periphery of the entirety of the internal lead 78b, specifically a portion of the internal lead 78b that is located inside the sensor element 101 (the portion interposed between the third substrate layer 3 and the first solid electrolyte layer 4), is covered by the lead insulation layer 79. The lead insulation layer 79 is connected to the rear end portion of the reference electrode insulation layer 43 and is formed integrally with the reference electrode insulation layer 43. Thus, the reference electrode 42 is electrically insulated from the first solid electrolyte layer 4, and the internal lead 78b is electrically insulated from both the third substrate layer 3 and the first solid electrolyte layer 4. The reference electrode insulation layer 43 and the lead insulation layer 79 are insulation layers formed from ceramics such as alumina. The entirety of the reference electrode insulation layer 43 and the lead insulation layer 79 is dense with a porosity of 5% or less, i.e., they are configured as a dense region. Such a configuration reduces the likelihood of the reference gas reaching the reference electrode 42 from the outside of the sensor element 101 via the lead insulation layer 79.

The heater section 70, as described above, includes the heater 72, the heater insulation layer 74, and the lead 76. The heater insulation layer 74 covers the surroundings of the heater 72 and the surroundings of the lead 76, except for the connection portion between the lead 76 and the through-hole 73. Thus, the heater 72 and the lead 76 are electrically insulated from the second substrate layer 2 and the third substrate layer 3. The heater insulation layer 74 is an insulation layer formed from ceramics such as alumina. The heater insulation layer 74 may be configured as a dense layer with a porosity of 5% or less, or as a porous layer with a porosity exceeding 5%.

The porosity of the above described parts, such as the reference electrode lead 78, lead insulation layer 79, and heater insulation layer 74, is defined as a value derived as follows by using an image (SEM image) obtained in an observation through a scanning electron microscope (SEM). First, a measurement object is cut to have a section. The section, regarded as an observation surface, is embedded with resin and is polished, whereby an observation sample is obtained. Subsequently, the observation surface of the observation sample is photographed by SEM photography (with a secondary electron image, an acceleration voltage of 15 kV, and a magnifying power of 1000, but a magnifying power higher than 1000 and lower than or equal to 5000 if the magnifying power of 1000 is inappropriate), whereby a SEM image of the measurement object is obtained.

Subsequently, the obtained image is analyzed, so that a threshold value is determined using the discriminant analysis method (Otsu binarization method) from a brightness distribution of brightness data on the pixels in the image. Then, each pixel in the image is binarized into an object section and a pore section based on the determined threshold value, and the area of the object section and the area of the pore section are calculated. Furthermore, the percentage of the area of the pore section relative to the overall area (i.e., the total area of the object section and the pore section) is derived as the porosity (in %).

The limiting current when oxygen is pumped from the surroundings of the reference electrode 42 to the surroundings of the outer pump electrode 23 is defined as a value measured as follows. First, the sensor element 101 is placed in the atmosphere, and the heater 72 is energized to heat the sensor element 101 to a predetermined operating temperature (for example, 800° C.). The variable power sources 25, 46, and 52 are all kept in a state where no voltage is applied. In this state, the voltage Vp3 is applied between the outer pump electrode 23 and the reference electrode 42 by the power source circuit 92 such that oxygen is pumped from the surroundings of the reference electrode 42 to the surroundings of the outer pump electrode 23. During this process, the pump current Ip3 flowing between the two electrodes 23 and 42 is measured. The voltage Vp3 is set as a direct current voltage. As the voltage Vp3 is gradually increased, the pump current Ip3 also rises gradually. However, when the voltage Vp3 exceeds a certain level, further increasing the voltage Vp3 does not raise the pump current Ip3, indicating that the pump current Ip3 has reached its upper limit. This upper limit of the pump current Ip3 is measured as the limiting current.

Now, an exemplary method of manufacturing the gas sensor 100 will be described. First, six non-calcinated ceramic green sheets each containing an oxygen-ion-conductive solid electrolyte, such as zirconia, as a ceramic component are prepared. In each of these green sheets, a plurality of sheet holes to be used for positioning during printing or stacking as well as necessary through-holes and the like are provided in advance. Furthermore, the green sheet that is to become the spacer layer 5 is preliminarily subjected to a punching process or the like in which a space that is to become the measurement-object gas flow section is provided. Then, a pattern-printing process and a drying process for forming various patterns in the ceramic green sheets are performed in correspondence with the first substrate layer 1, the second substrate layer 2, the third substrate layer 3, the first solid electrolyte layer 4, the spacer layer 5, and the second solid electrolyte layer 6. Specific patterns to be formed include, for example, patterns that serve as electrodes such as the reference electrode 42 described above, leads connected to the electrodes, such as the internal lead 78b, lead insulation layer 79, the connector electrodes 71, and the heater section 70. The pattern-printing process is performed in which a pattern-forming paste prepared in accordance with the properties required for an object to be formed is applied onto a green sheet by using a known screen printing technique. The pattern for the lead insulation layer 79 may be formed by separating the portion that covers the underside of the internal lead 78b from the portion that covers the upper side. Similarly, the pattern for the heater insulation layer 74 of the heater section 70 may be formed by separating the portion that covers the underside of the heater 72 and the lead 76 from the portion that covers the upper side. After performing the pattern-printing process, a drying process is performed by using known a known drying technique. When the pattern-printing process and drying process are completed, a printing process for a bonding paste, used to stack and bond the green sheets corresponding to the respective layers, are performed. Subsequently, the green sheets provided with the bonding paste are stacked in a predetermined order while being positioned at the sheet holes, and are then subjected to a pressure bonding process in which the green sheets are put under predetermined temperature and pressure conditions to be pressure bonded into a single layered body. The layered body thus obtained contains a plurality of sensor elements 101 therein. The layered body is cut into pieces each having the size of the sensor element 101. Subsequently, a pattern that is to become the peripheral-face lead 78a is formed by screen printing on a section obtained by cutting the layered body, i.e., on a part that serves as the right face of the sensor element 101. Furthermore, the pattern is dried. Then, the cut layered body is calcinated at a predetermined calcination temperature, whereby a sensor element 101 is obtained. Furthermore, the pattern-forming paste for forming the conducting section 77 (here, the connector electrode 71a and the reference electrode lead 78), particularly for the internal lead 78b, is a paste containing particles of noble metal or high-melting-point metal, as described above. The pattern-forming paste for forming the lead insulation layer 79 and the heater insulation layer 74 is a paste containing particles of ceramics such as alumina, as described above.

Here, the limiting current when oxygen is pumped from the surroundings of the reference electrode 42 to the surroundings of the outer pump electrode 23, as well as the diffusion resistance of the internal lead 78b, are adjustable by adjusting the shape and porosity of the internal lead 78b. This is because, as described above, the diffusion resistance of the internal lead 78b is based on its length in the extending direction, width, thickness, and porosity. The porosity of the internal lead 78b is adjustable, for example, by adjusting particle diameters of particles contained in the pattern-forming paste for forming the internal lead 78b, adjusting a particle diameter and content ratio of the pore-forming material, or adjusting a calcination temperature and calcination time when the layered body is calcinated. The same considerations can be applied to the adjustment of the porosity of the lead insulation layer 79 and the heater insulation layer 74.

Subsequently, a gas sensor 100 having the sensor element 101 incorporated therein is manufactured. For example, an element sealing unit 141 is attached to the sensor element 101 to seal and secure the sensor element 101. Furthermore, a connector 150 and lead wires 155 are attached to the rear end of the sensor element 101 in such a manner as to be electrically continuous with the connector electrodes 71. Additionally, a protection cover 130 is attached to a part of the element sealing unit 141 that is near the front end of the sensor element 101. Furthermore, an outer cylinder 148 is attached to a part of the element sealing unit 141 that is near the rear end of the sensor element 101, the lead wires 155 are routed from the outer cylinder 148 to the outside. Then, a control device 95 and the sensor element 101 are connected to each other via the lead wires 155. Thus, a gas sensor 100 is obtained.

Now, the processing performed by the controller 96 when the gas sensor 100 detects the NOx concentration in the measurement-object gas will be described. First, the CPU 97 of the controller 96 starts the operation of the sensor element 101. Specifically, the CPU 97 sends a control signal to the heater power source to heat the sensor element 101 by the heater 72. At this time, the CPU 97 heats the sensor element 101 to a predetermined operating temperature (for example, 800° C.). Next, the CPU 97 starts the control of the pump cells 21, 41, 50, and 90 described above, and the acquisition of the voltages V0, V1, V2, and Vref from the sensor cells 80 to 83 described above. Under this condition, when the measurement-object gas is introduced through the gas inlet 10, the measurement-object gas passes through the first diffusion control section 11, the buffer space 12, and the second diffusion control section 13, and reaches the first internal cavity 20. Next, the oxygen concentration of the measurement-object gas is adjusted in the first internal cavity 20 and the second internal cavity 40 by the main pump cell 21 and the auxiliary pump cell 50. The adjusted measurement-object gas then reaches the third internal cavity 61. The CPU 97 detects the NOx concentration in the measurement-object gas based on the acquired pump current Ip2 and the correspondence relationship stored in the storage unit 98.

Here, the measurement-object gas is introduced into the measurement-object gas flow section, such as through the gas inlet 10, of the sensor element 101 from the sensor element chamber 133 illustrated in FIG. 1. On the other hand, as the reference electrode lead 78 (the peripheral-face lead 78a and the internal lead 78b) is porous with a porosity exceeding 5%, the reference gas is introduced to the reference electrode 42 from the outside of the sensor element 101 (the space 149 illustrated in FIG. 1) via the reference electrode lead 78. In the present embodiment, the sensor element 101 is designed such that the limiting current when oxygen is pumped from the surroundings of the reference electrode 42 to the surroundings of the outer pump electrode 23 is 0.3 μA or more. This ensures that the diffusion resistance of the reference electrode lead 78, particularly the internal lead 78b, is at or below a predetermined value (i.e., not excessively high). Such a configuration facilitates the maintenance of the oxygen concentration around the reference electrode 42 at an appropriate level compared to a case where the diffusion resistance of the internal lead 78b is excessively high, thereby reducing the decrease in detection accuracy of the specific gas concentration. The limiting current is preferably 0.5 μA or more. The porosity of the internal lead 78b is preferably 10% or more. The porosity of the internal lead 78b is 40% or less. Such a configuration makes the internal lead 78b less likely to become disconnected.

Additionally, during the non-operational period of the sensor element 101, the internal lead 78b and the lead insulation layer 79 may adsorb water from the outside the sensor element 101, specifically from the space 149. The water in the space 149 may be present in small amounts initially or may enter through the gap between the rubber stopper 157 and the outer cylinder 148. When the controller 96 starts the operation of the sensor element 101, the heater 72 heats the sensor element 101, causing the water in the internal lead 78b and the lead insulation layer 79 to vaporize and escape to the outside of the sensor element 101, specifically into the space 149. However, until the water has been sufficiently drained, the presence of vaporized water may reduce the oxygen concentration around the reference electrode 42. Therefore, the time required for the water to drain from the internal lead 78b and the lead insulation layer 79 to the outside of the sensor element 101 (hereinafter referred to as “water drain time”) affects the time from the start of the operation of the sensor element 101 until the potential of the reference electrode 42 stabilizes (hereinafter referred to as “stabilization time”). In the present embodiment, the entirety of the lead insulation layer 79 is configured to be dense (as a dense region). Such a configuration reduces the amount of water (water vapor) adsorbed by the lead insulation layer 79 during the non-operational period of the sensor element 101, compared to a case where at least a portion of the lead insulation layer 79 is configured to be porous with a porosity exceeding 5% (as a porous region). In the present embodiment, the internal lead 78b is a conductor mainly composed of a noble metal or a high-melting-point metal. The noble metal or high-melting-point metal used for the internal lead 78b generally has properties that make it less likely to adsorb water compared to alumina used for the lead insulation layer 79, thus, the internal lead 78b is less likely to adsorb water than the lead insulation layer 79. Such a configuration reduces the amount of water (water vapor) adsorbed by the internal lead 78b during the non-operational period of the sensor element 101. As the amount of water adsorbed by both the lead insulation layer 79 and the internal lead 78b is reduced during this non-operational period of the sensor element 101, the water drain time is shortened, which in turn shortens the stabilization time.

Here, the correspondence between the elements according to the present embodiment and the elements according to the present invention is clarified. The first substrate layer 1, the second substrate layer 2, the third substrate layer 3, the first solid electrolyte layer 4, the spacer layer 5, and the second solid electrolyte layer 6 according to the present embodiment each correspond to the solid electrolyte layer according to the present invention, and these layers correspond to the element body. Additionally, the reference electrode 42 corresponds to the reference electrode, the outer pump electrode 23 corresponds to the measurement-object gas side electrode, the connector electrode 71a corresponds to the reference electrode terminal, the reference electrode lead 78 corresponds to the reference electrode lead, and the conducting section 77 corresponds to the first conducting section. Furthermore, the lead insulation layer 79 corresponds to the lead insulation layer.

In the sensor element 101 included in the gas sensor 100 according to the present embodiment described in detail above, the reference electrode lead 78 allows the reference gas to flow from the outside of the sensor element 101 to the reference electrode 42. The limit current, when oxygen is pumped out from the surroundings of the reference electrode 42 to the surroundings of the outer pump electrode 23, is 0.3 μA or more. The entirety of the lead insulation layer 79 is configured to be dense (as a dense region) with a porosity of 5% or less. As the entirety of the lead insulation layer 79 is configured to be dense (as a dense region), the amount of water (water vapor) adsorbed by the lead insulation layer 79 during the non-operational period of the sensor element 101 is reduced compared to a case where at least a portion of the lead insulation layer 79 is configured to be porous. Such a configuration shortens the time from the start of the operation of the sensor element 101 until the water drains out of the sensor element 101 (water drain time), thereby shortening the time from the start of the operation of the sensor element 101 until the potential of the reference electrode 42 stabilizes (stabilization time). Furthermore, as the limiting current is 0.3 μA or more, indicating that the diffusion resistance of the reference electrode lead 78, particularly the internal lead 78b, is at or below a predetermined value (not excessively high), the maintenance of the oxygen concentration around the reference electrode 42 at an appropriate level is facilitated compared to a case where the diffusion resistance of the reference electrode lead is excessively high, thereby reducing the decrease in detection accuracy of the specific gas concentration (NOx concentration).

The present invention is not limited whatsoever to the above embodiment, and various embodiments are conceivable so long as they belong to the technical scope of the present invention.

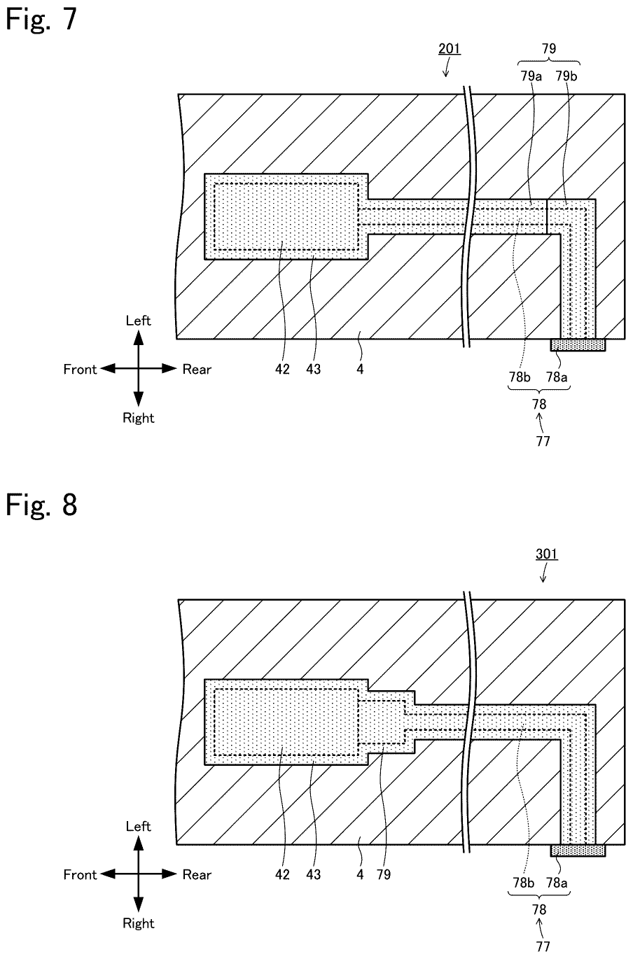

For example, in the embodiment described above, the entirety of the lead insulation layer 79 is configured to be dense (as the dense region). However, as illustrated in a sensor element 201 according to a modification in FIG. 7, the lead insulation layer 79 may be configured such that a region of 50% or more but less than 100%, from the end on the reference electrode 42 side, of the total area of the lead insulation layer 79 in the planar view is a dense region 79a with the porosity of 5% or less, while the remaining region is a porous region 79b. The total area of the lead insulation layer 79 in the planar view refers to the total area of the lead insulation layer 79 as seen from above or below, or in other words, it refers to the projected area of the lead insulation layer 79 onto the upper or lower face of the sensor element 201. A configuration in which a region of 80% or more, from the end on the reference electrode 42 side, of the total area of the lead insulation layer 79 in the planar view is configured as the dense region is preferable. In a case where a portion region of the total area of the lead insulation layer 79 in the planar view is configured as the dense region, while the remaining region is configured as the porous region, as the region that includes at least the end on the reference electrode 42 side of the lead insulation layer 79 is configured as the dense region, even if the lead insulation layer 79 adsorbs water during the non-operational period of the sensor element 101, the water drains more easily from the sensor element 101 after it starts operating, compared to a case where the porous region is provided on the reference electrode 42 side relative to the dense region in the lead insulation layer 79. Consequently, such a configuration shortens the water drain time, thereby shortening the stabilization time.

In the embodiment described above, a cross-sectional area and porosity of the internal lead 78b of the reference electrode lead 78 are approximately constant overall. However, as illustrated in a sensor element 301 in FIG. 8, the cross-sectional area of the internal lead 78b may be larger on the reference electrode 42 side compared to the peripheral-face lead 78a side (connector electrode 71a side). Additionally, or alternatively, the porosity of the internal lead 78b may be greater on the reference electrode 42 side compared to the peripheral-face lead 78a. As the cross-sectional area and/or porosity of the internal lead 78b on the reference electrode 42 side becomes larger compared to that of the peripheral-face lead 78a side, the diffusion resistance of the internal lead 78b on the reference electrode 42 side becomes smaller than that of the peripheral-face lead 78a side. Such a configuration increases the likelihood of oxygen accumulating around the reference electrode 42, thereby facilitating the maintenance of the oxygen concentration around the reference electrode at the appropriate level. Consequently, the decrease in the detection accuracy of the specific gas concentration (NOx concentration) is reduced.

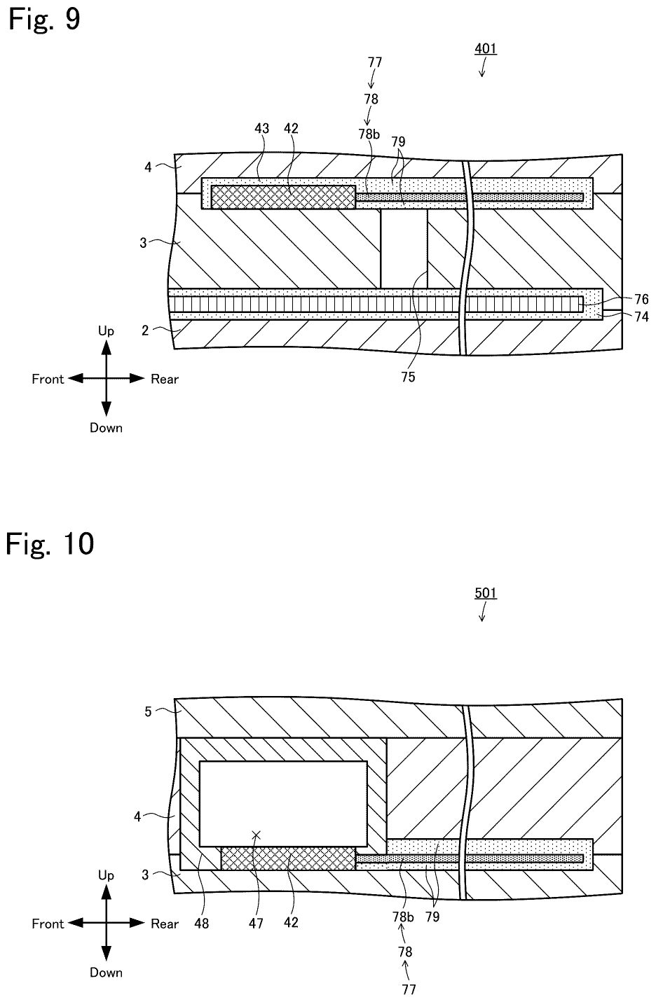

In the embodiment described above, the heater section 70 includes the heater 72, the heater insulation layer 74, and the lead 76, and the lead insulation layer 79 and the heater insulation layer 74 are independently provided such that they do not connect with each other inside the sensor element 101. However, as illustrated in a sensor element 401 in FIG. 9, the heater section 70 may further include a pressure relief hole (communication hole) 75. The pressure relief hole 75 is provided to penetrate through the third substrate layer 3, and connects the lead insulation layer 79 and the heater insulation layer 74. This pressure relief hole 75 is provided for the purpose of alleviating the increase in internal pressure resulting from the rise in temperature within the heater insulation layer 74. In a case where the heater section 70 includes the pressure relief hole 75, the entirety of the heater insulation layer 74 is configured to be dense with the porosity of 5% or less. As the heater section 70 includes a pressure relief hole 75 and the entirety of the heater insulation layer 74 is configured to be dense (as a dense region), the amount of water adsorbed by the heater insulation layer 74 via the lead insulation layer 79 and the pressure relief hole 75 during the non-operational period of the sensor element 101 is reduced.

In the embodiment described above, the reference electrode insulation layer 43 is configured to be dense along with the entirety of the lead insulation layer 79. However, the reference electrode insulation layer 43 may also be configured to be porous.

In the embodiment described above, the upper, front, rear, left, and right faces of the reference electrode 42 are covered by the reference electrode insulation layer 43. However, as illustrated in a sensor element 501 in FIG. 10, a reference gas chamber 47 may be provided inside the sensor element 101 as a space for accumulating the reference gas, and the reference electrode 42 may be disposed inside this reference gas chamber 47. In the sensor element 501 illustrated in FIG. 10, the reference gas chamber 47 is an internal space provided inside the sensor element 501 by hollowing out the first solid electrolyte layer 4. Specifically, it is the space surrounded by the lower surface of the spacer layer 5, the internal front, rear, left, and right side faces of the first solid electrolyte layer 4, and the upper face of the third substrate layer 3. A dense layer 48 is disposed inside the reference gas chamber 47. The dense layer 48 is a dense layer with a porosity of 5% or less, covering the inner peripheral face of the reference gas chamber 47. Additionally, the dense layer 48 does not cover the upper face of the reference electrode 44. Thus, the reference electrode 42 is exposed to the space surrounded by the dense layer 48 inside the reference gas chamber 47. Furthermore, the dense layer 48 also covers the portion of the internal lead 78b that is located inside the reference gas chamber 47. Therefore, by being able to accumulate the reference gas in the reference gas chamber 47, the variation in the oxygen concentration around the reference electrode 42 is further reduced. Instead of having the dense layer 48 disposed inside the reference gas chamber 47, a porous body may be filled inside the reference gas chamber 47, or neither the dense layer 48 nor the porous body may be provided inside the reference gas chamber 47.

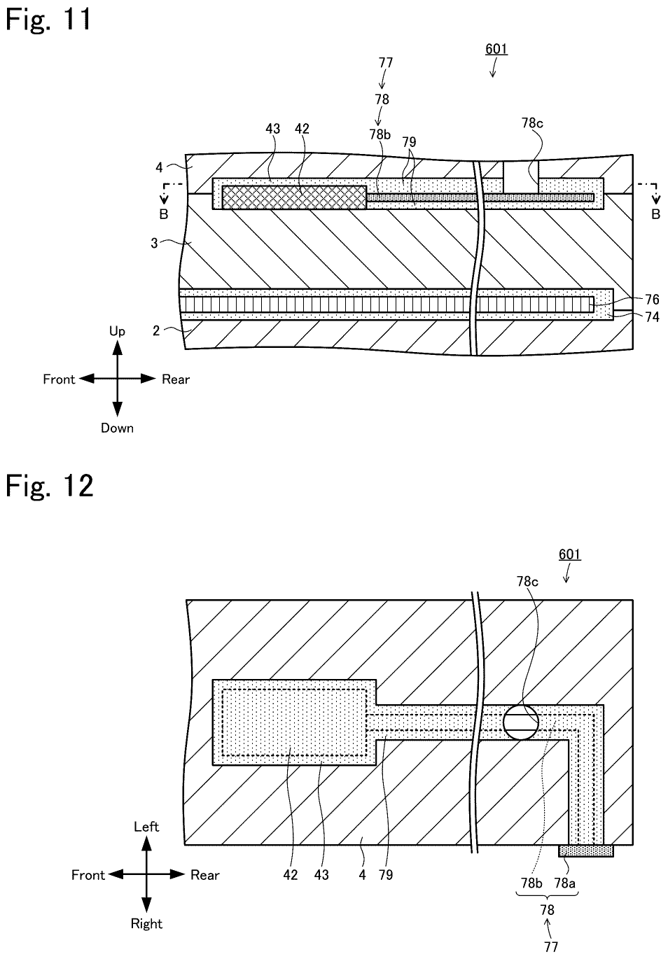

In the embodiment described above, the outer circumference of the portion of the internal lead 78b that is located inside the sensor element 101 (the portion interposed between the third substrate layer 3 and the first solid electrolyte layer 4) is covered by the lead insulation layer 79. However, as illustrated in a sensor element 601 in FIGS. 11 and 12, a portion of the outer circumference of the portion of the internal lead 78b that is located inside the sensor element 101 (a part in the extension direction of the internal lead 78b) may be exposed inside a preliminary chamber 78c. FIG. 11 is a partially enlarged cross-sectional view of an area around a reference electrode 42, a reference electrode lead 78 and a lead 76 of the sensor element 601. FIG. 12 is a cross-sectional view taken along B-B in FIG. 11. The preliminary chamber 78c is a space formed in the element body (layered body) of the sensor element 601. In the examples illustrated in FIGS. 11 and 12, the preliminary chamber 78c is a cylindrical space formed as a hole that penetrates the first solid electrolyte layer 4 vertically. As the portion of the internal lead 78b is exposed inside the preliminary chamber 78c, in other words, as the preliminary chamber 78c is in contact with the portion of the internal lead 78b, the reference gas flowing through the internal lead 78b diffuses into the preliminary chamber 78c, which has a smaller diffusion resistance than the internal lead 78b. This allows the preliminary chamber 78c to function as a buffer for variations in the oxygen concentration in the reference gas. Additionally, the preliminary chamber 78c may have a shape other than cylindrical, for example, it may have a shape of a rectangular prism. Furthermore, there may be multiple preliminary chambers 78c instead of just one. In the examples illustrated in FIGS. 11 and 12, the upper face of the internal lead 78b is exposed inside the preliminary chamber 78c, however, multiple faces of the internal lead 78b may be exposed inside the preliminary chamber 78c as the internal lead 78b passes through the preliminary chamber 78c.

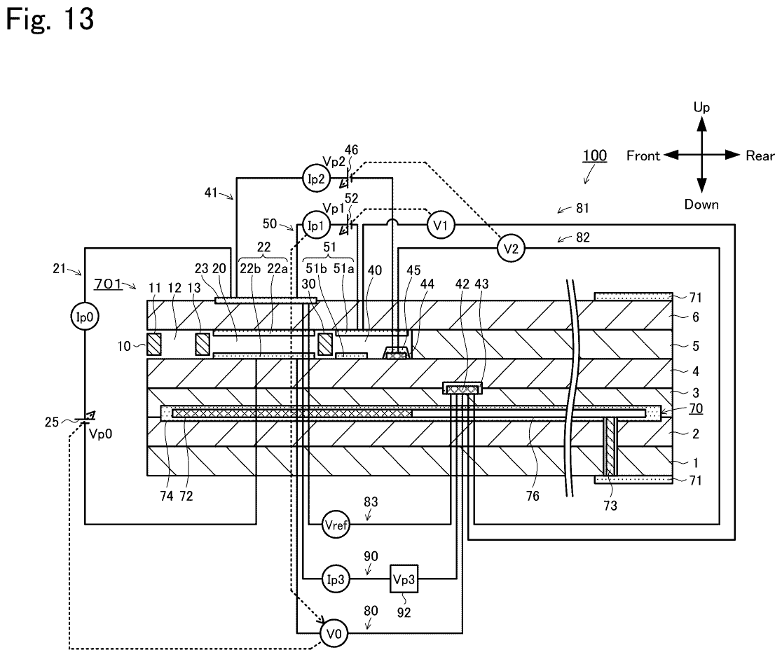

In the embodiment described above, the sensor element 101 of the gas sensor 100 includes the first internal cavity 20, the second internal cavity 40, and the third internal cavity 61. However this is not limiting. For example, as illustrated in a sensor element 701 according to yet another modification in FIG. 13, the sensor element 701 may not include the third internal cavity 61. In the sensor element 701 according to yet another modification in FIG. 13, between the lower face of the second solid electrolyte layer 6 and the upper face of the first solid electrolyte layer 4 are provided a gas inlet 10, a first diffusion control section 11, a buffer space 12, a second diffusion control section 13, a first internal cavity 20, a third diffusion control section 30, and a second internal cavity 40 that are next to one another and connect with one another in that order. Additionally, the measurement electrode 44 is disposed on the upper face of the first solid electrolyte layer 4 inside the second internal cavity 40. The measurement electrode 44 is covered by the fourth diffusion control section 45. The fourth diffusion control section 45 is a membrane made of a ceramic porous body such as alumina (Al₂O₃). As with the fourth diffusion control section 60 in the embodiment described above, the fourth diffusion control section 45 has a role of limiting the amount of NOx flowing into the measurement electrode 44. Additionally, the fourth diffusion control section 45 also functions as a protective film for the measurement electrode 44. The ceiling electrode portion 51a of the auxiliary pump electrode 51 is formed directly above the measurement electrode 44. Even with such a configuration of the sensor element 701, the NOx concentration can be detected by the measurement pump cell 41, as with the embodiment described above. In the sensor element 701 illustrated in FIG. 13, the surroundings of the measurement electrode 44 functions as a measurement chamber. In other words, the surroundings of the measurement electrode 44 has a role as that of the third internal cavity 61.

In the sensor element 101 described above, the circuit of the reference gas adjustment pump cell 90 may be omitted, or the gas sensor 100 may not include the power source circuit 92. Additionally, the gas sensor 100 may not include the control device 95. For example, the gas sensor 100 may be include an external connection connector attached to the lead wires 155 to connect the control device 95 and the lead wires 155 instead of including the control device 95.

In the sensor element 101 described above, the surface of the front side of the sensor element 101, which includes the outer pump electrode 23 and is exposed to the sensor element chamber 133, may be covered with a porous protective layer made of ceramics such as alumina.

In the embodiment described above, the CPU 97 performs feedback control of the voltage Vp2 of the variable power source 46 such that the voltage V2 reaches the target value V2*, and detects the NOx concentration in the measurement-object gas based on the detected value (pump current Ip2) at that time. However, this is not limiting. For example, the CPU 97 may control the measurement pump cell 41 (for example, control the voltage Vp2) such that the pump current Ip2 reaches a constant target value Ip2*, and may detect the NOx concentration based on the detected value (the voltage V2) at that time. As the measurement pump cell 41 is controlled such that the pump current Ip2 reaches the target value Ip2*, oxygen is drawn out from the third internal cavity 61 at a nearly constant flow rate. Consequently, the oxygen concentration in the third internal cavity 61 changes according to the amount of oxygen generated by the reduction of NOx in the measurement-object gas. This, in turn, leads to variations in the voltage V2. Therefore, the voltage V2 corresponds to the NOx concentration in the measurement-object gas. As a result, the controller 96 can calculate the NOx concentration based on this voltage V2. In this case, for example, the relationship between the voltage V2 and the NOx concentration can be stored in advance in the storage unit 98.

In the embodiment described above, the sensor element 101 is configured to detect the NOx concentration in the measurement-object gas. However, it is not limited to this and may be used to detect the concentration of any specific gas in the measurement-object gas. For example, it may be used to detect the concentration of other oxides as specific gas concentrations, not just NOx. In the case where the specific gas is an oxide, as in the embodiment described above, oxygen is generated when the specific gas itself is reduced in the third internal cavity 61. Therefore, the measurement pump cell 41 acquires the detected value (for example, pump current Ip2) corresponding to this oxygen to detect the concentration of the specific gas. The specific gas may also be a non-oxide, such as ammonia. In the case where the specific gas is a non-oxide, converting the specific gas into an oxide (for example, converting ammonia to NO) generates oxygen when the converted gas is reduced in the third internal cavity 61. Therefore, the measurement pump cell 41 acquires the detected value (for example, pump current Ip2) corresponding to this oxygen to detect the concentration of the specific gas. For example, the inner pump electrode 22 of the first internal cavity 20 functions as a catalyst, allowing ammonia to be converted into NO within the first internal cavity 20.

In the embodiment described above, the element body of the sensor element 101 is configured as the layered body having multiple solid electrolyte layers (layers 1 to 6). However, this is not limiting. The element body of the sensor element 101 should include at least one oxygen-ion-conductive solid electrolyte layer. For example, in FIG. 2, layers 1 to 5, other than the second solid electrolyte layer 6, may consist of layers made from materials other than solid electrolyte layers (for example, layers made of alumina). In this case, each electrode of the sensor element 101 may be disposed on the second solid electrolyte layer 6. For example, the measurement electrode 44 in FIG. 2 may be arranged on the lower face of the second solid electrolyte layer 6. Additionally, the reference electrode 42 may be provided behind the measurement-object gas flow section and on the lower face of the second solid electrolyte layer 6.

In the embodiment described above, the inner pump electrode 22 is configured as a cermet electrode made of Pt containing 1% Au and ZrO₂. However, this is not limiting. The inner pump electrode 22 should contain a noble metal with catalytic activity (for example, at least one of Pt, Rh, Ir, Ru, or Pd) and a noble metal with catalytic activity reduction ability (for example, Au) that reduces the catalytic activity of the noble metal with catalytic activity for the specific gas. The auxiliary pump electrode 51 should contain a noble metal with catalytic activity and a noble metal with catalytic activity reduction ability, as with the inner pump electrode 22. The outer pump electrode 23, reference electrode 42, and measurement electrode 44 should each contain a noble metal with catalytic activity as described above. Each of the electrodes 22, 23, 42, 44, and 51 is preferably a cermet containing a noble metal with catalytic activity and an oxide with oxygen ion conductivity (for example, Zro₂), but one or more of these electrodes may not be cermets. Each of the electrodes 22, 23, 42, 44, and 51 is preferably a porous body, but one or more of these electrodes may not be porous bodies.

In the embodiment described above, the outer pump electrode 23 serves as multiple electrodes, including an outer main pump electrode that is part of the main pump cell 21 and is disposed on the portion exposed to the measurement-object gas outside the sensor element 101, an outer auxiliary pump electrode that is part of the auxiliary pump cell 50 and is disposed on the portion exposed to the measurement-object gas outside the sensor element 101, an outer measurement electrode that is part of the measurement pump cell 41 and is disposed on the portion exposed to the measured gas outside the sensor element 101, and the measurement-object gas side electrode that is part of the reference gas adjustment pump cell 90 and is disposed on the portion exposed to the measured gas outside the sensor element 101. However, this is not limiting. One or more of the outer main pump electrode, the outer auxiliary pump electrode, the outer measurement electrode, and the measurement-object gas side electrode may be provided separately from the outer pump electrode 23, on the outside of the sensor element 101.

In the embodiment described above, the controller 96 sets the target value V0* for the voltage V0 based on the pump current Ip1 such that the pump current Ip1 reaches the target value Ip1* (feedback control), and feedback controls the pump voltage Vp0 such that the voltage V0 reaches the target value V0*. However, other control methods may also be used. For example, the controller 96 may feedback control the pump voltage Vp0 based on the pump current Ip1 such that the pump current Ip1 reaches the target value Ip1*. In other words, the controller 96 may omit the acquisition of the voltage V0 from the main-pump-control oxygen-partial-pressure detection sensor cell 80 and the setting of the target value V0*, and directly control the pump voltage Vp0 (and thus control the pump current Ip0) based on the pump current Ip1.

In the embodiment described above, the pump current Ip3 is a constant direct current, however this is not limiting. For example, the pump current Ip3 may be a pulsed, intermittent current. Additionally, in the embodiment described above, the pump current Ip3 is a constant direct current that always flows in the direction of drawing oxygen into the area around the reference electrode 42, this is not limiting. For example, there may be a period during which the pump current Ip3 flows in the direction of pumping oxygen out from the surroundings of the reference electrode 42. Even in such a case, the overall movement of oxygen, when observed over a sufficiently long predetermined period, may be in the direction of pumping oxygen into the surroundings the reference electrode 42.

### EXAMPLES

Specifically fabricated examples of the sensor element will now be described. Note that the present invention is not limited to the following examples.

### Example 1

Example 1 was obtained by fabricating the gas sensor 100 illustrated in FIGS. 1 to 6 using the manufacturing method described above. In fabricating the sensor element 101, ceramic green sheets were each obtained by mixing zirconia particles having 4 mol % of yttria added thereto as a stabilizer with an organic binder and an organic solvent, and then molding the mixture by tape molding. The reference electrode 42 was made as a porous cermet electrode composed of Pt and zirconia. The pattern for the reference electrode 42 was formed using a paste created by mixing Pt powder, zirconia powder, a binder, and a pore-forming agent. The conducting section 77 was made as a porous cermet mainly composed of Pt, along with zirconia. The pattern for the conducting section 77 was formed using a paste created by mixing Pt powder, zirconia powder, a binder, and a pore-forming agent. The paste for the lead insulation layer 79 was prepared by mixing alumina powder and a binder solution in a weight ratio of 1:2. The porosity of the internal lead 78b in the conducting section 77 was 15%. The ratio of the dense region, from the end on the reference electrode 42 side, to the total area of the lead insulation layer 79 in a top view was 100%. The limiting current when oxygen is pumped from the surroundings of the reference electrode 42 to the surroundings of the outer pump electrode 23 was 1.0 μA. The measurement of porosity was conducted using image analysis based on the SEM images described above. The measurement of the limiting current was performed using the method described above.

### Example 2

Except for setting the limiting current to 2.0 μA, a gas sensor 100 identical to Example 1 was produced for Example 2. The limiting current was changed by altering the shape of the internal lead 78b (specifically, the length in the extending direction) compared to Example 1.

### Example 3

Except for setting the porosity of the internal lead 78b to 6%, setting the ratio of the dense region, from the end on the reference electrode 42 side, to the total area of the lead insulation layer 79 in a top view to 50%, and setting the limiting current to 0.3 μA, a gas sensor 100 identical to Example 1 was produced for Example 3. The paste for the porous region of the lead insulation layer 79 was prepared by further mixing a pore-forming agent into the paste used for the lead insulation layer 79 in Example 1 (the same applies to Examples 4 to 8 and Comparative Example 2 described later). The limiting current was changed by altering the shape of the internal lead 78b (specifically, the length in the extending direction) and the porosity compared to Example 1.

### Example 4

Except setting the ratio of the dense region, from the end on the reference electrode 42 side, to the total area of the lead insulation layer 79 in a top view to 80%, and setting the limiting current to 0.7 μA, a gas sensor 100 identical to Example 1 was produced for Example 4. The limiting current was changed by altering the shape of the internal lead 78b (specifically, the length in the extending direction) compared to Example 1.

### Example 5

Except for setting the porosity of the internal lead 78b to 10%, setting the ratio of the dense region, from the end on the reference electrode 42 side, to the total area of the lead insulation layer 79 in a top view to 70%, and setting the limiting current to 0.5 μA, a gas sensor 100 identical to Example 1 was produced for Example 5. The limiting current was changed by altering the shape of the internal lead 78b (specifically, the length in the extending direction) and the porosity compared to Example 1.

### Example 6

Except for setting the porosity of the internal lead 78b to 10%, setting the ratio of the dense region, from the end on the reference electrode 42 side, to the total area of the lead insulation layer 79 in a top view to 90%, and setting the limiting current to 0.5 μA, a gas sensor 100 identical to Example 1 was produced for Example 6. The limiting current was changed by altering the length in the extending direction and the porosity compared to Example 1.

### Example 7

Except for setting the porosity of the internal lead 78b to 8%, setting the ratio of the dense region, from the end on the reference electrode 42 side, to the total area of the lead insulation layer 79 in a top view to 75%, and setting the limiting current to 0.4 μA, a gas sensor 100 identical to Example 1 was produced for Example 7. The limiting current was changed by altering the shape of the internal lead 78b (specifically, the length in the extending direction) and the porosity compared to Example 1.

### Example 8

Except for setting the porosity of the internal lead 78b to 10%, setting the ratio of the dense region, from the end on the reference electrode 42 side, to the total area of the lead insulation layer 79 in a top view to 80%, and setting the limiting current to 0.7 μA, a gas sensor 100 identical to Example 1 was produced for Example 8. The limiting current was changed by altering the porosity of the internal lead 78b compared to Example 1.

### Example 9

Except for setting the porosity of the internal lead 78b to 40%, and setting the limiting current to 2.0 μA, a gas sensor 100 identical to Example 1 was produced for Example 9. The limiting current was changed by altering the shape of the internal lead 78b (specifically, the length in the extending direction) and the porosity compared to Example 1.

### Example 10

A gas sensor 100 was produced, which includes a sensor element 601 with a preliminary chamber 78c (see FIGS. 11 and 12), instead of a sensor element 101 without a preliminary chamber (see FIGS. 1 to 6), for Example 10. The sensor element 601 was produced using a manufacturing method similar to Example 1, except for the following points: a space (hole) for the preliminary chamber 78c was pre-punched into the green sheet that serves as the first solid electrolyte layer 4, and the shape of the pattern covering the upper side of the internal lead 78b in the lead insulation layer 79 included a space (hole) for the preliminary chamber 78c. The porosity of the internal lead 78b was set to 15%, the ratio of the dense region, from the end on the reference electrode 42 side, to the total area of the lead insulation layer 79 in a top view was set to 100%, and the limiting current was set to 2.0 μA. The limiting current was changed compared to Example 1 by incorporating the preliminary chamber 78c.

### Comparative Example 1

Except for setting the porosity of the internal lead 78b to 5%, and setting the limiting current to 2.0 μA, a gas sensor 100 identical to Example 1 was produced for Comparative Example 1. The limiting current was changed by altering the shape of the internal lead 78b (specifically, the length in the extending direction) and the porosity compared to Example 1.

### Comparative Example 2

Except for setting the ratio of the dense region, from the end on the reference electrode 42 side, to the total area of the lead insulation layer 79 in a top view to 40%, a gas sensor 100 identical to Example 1 was produced for Comparative Example 2.

### Evaluation Test 1: Evaluation of Stabilization Time

The gas sensor 100 according to Example 1 was stored in a constant temperature and humidity chamber at 40° C. and 85% humidity for one week, allowing water to be adsorbed by the internal lead 78b and the lead insulation layer 79. Next, the gas sensor 100 according to Example 1 was attached to a pipe. A model gas was prepared with nitrogen as the base gas, an oxygen concentration of 0%, and an NOx concentration of 1500 ppm, which was then flowed through the pipe as the measurement-object gas. In this state, the sensor element 101 was operated by the control device 95. Specifically, the control device 95 energized the heater 72 to heat the sensor element 101, maintaining its temperature at 800° C. Additionally, the control device 95 was in the state of continuously controlling each of the pump cells 21, 41, and 50 described above, and acquiring the respective voltages V0, V1, V2, and Vref from each of the sensor cells 80 to 83 described above. The reference gas adjustment pump cell 90 was not allowed to operate. The above condition was maintained for 60 minutes from the start of the operation of the sensor element 101 (heating start), during which the pump current Ip2 was continuously measured. The value of the pump current Ip2 after 60 minutes from the start of measurement was set as the reference value (100%), and the rate of change of the pump current Ip2 after 10 minutes form the start of measurement with respect to the reference value was calculated. The rate of change of the pump current Ip2 was similarly calculated for the gas sensors 100 according to Examples 2 to 10 and Comparative Examples 1 and 2.

Here, during the period from the start of the operation of the sensor element 101 until the water in the internal lead 78b and the lead insulation layer 79 has been sufficiently drained, the presence of vaporized water reduces the oxygen concentration around the reference electrode 42, leading to an unstable potential at the reference electrode 42. Therefore, until the potential of the reference electrode 42 stabilizes, the pump current Ip2 will not be stable, even if the NOX concentration of the measurement-object gas remains constant. A smaller rate of change in the pump current Ip2 indicates that the water has been sufficiently drained from the internal lead 78b and the lead insulation layer 79, and the potential of the reference electrode 42 is considered to be stable at that point, 10 minutes after the start of measurement. Therefore, the duration of stabilization time, which is the time from the start of the operation of the sensor element 101 until the potential of the reference electrode 42 stabilizes, can be evaluated based on the magnitude of the rate of change. When the calculated rate of change is 3% or less, the stabilization time was evaluated as short (denoted as “A”). When the calculated rate of change is more than 3% and 5% or less, the stabilization time was evaluated as short (denoted as “B”). When the calculated rate of change exceeds 5%, the stabilization time was evaluated as long (denoted as “F”).

### Evaluation Test 2: Evaluation of NOx Detection Accuracy

The gas sensor 100 according to Example 1 was attached to a pipe. The heater 72 was energized to maintain its temperature at 850° C., and the sensor element 101 was heated. Additionally, the reference gas adjustment pump cell 90 was operated. The control voltage Vp3 applied by the power source circuit 92 of the electrochemical reference gas adjustment pump cell 90 was a pulse voltage with a period T of 10 ms, an on time Ton of 2.0 ms, and an off time Toff of 8.0 ms (duty cycle 20%). The control voltage Vp3 applied by the power source circuit 92 was set such that the peak value of the control current Ip3 flowing during the voltage on time was 10 μA. The effective value Ip3ef of the control current Ip3 was 2 μA. In this state, a model gas was prepared with nitrogen as the base gas, an oxygen concentration of 10%, and an NOx concentration of 500 ppm, which was then flowed through the pipe as the measurement-object gas. This condition was maintained for 20 minutes, during which the voltage Vref was measured during this time. The same measurements were performed for Examples 2 to 8 and Comparative Examples 1 and 2.

The lower the oxygen concentration around the reference electrode 42 is compared to the oxygen concentration at the start of measurement (which is equal to the oxygen concentration in the atmosphere), the more the voltage Vref tends to decrease over time from the value at the start of measurement. Furthermore, the lower the voltage Vref is, the larger the pump current Ip2 tends to be compared to the correct value (the value corresponding to an NOx concentration of 500 ppm). Therefore, when the value of the voltage Vref at the start of measurement was set at 100%, and the measured voltage Vref remained within the predetermined range (80% or more) even after 20 minutes, the change (decrease) in the oxygen concentration around the reference electrode 42 was very small and the detection accuracy of the NOx concentration was evaluated as very high (denoted as “A”). When the measured voltage Vref remained within the predetermined range until 15 minutes elapsed but fell below the lower limit of the range before 20 minutes elapsed, the oxygen around the reference electrode 42 was slightly low, but the detection accuracy of the NOx concentration was evaluated as high (denoted as “B”). When the measured voltage Vref fell below the lower limit of the range before 15 minutes elapsed, the oxygen around the reference electrode 42 was insufficient and the detection accuracy of the NO concentration was evaluated as low (denoted as “F”).

For each of Examples 1 to 10 and Comparative Examples 1 and 2, the porosity of the internal lead 78b, the ratio of the dense region from the end on the reference electrode 42 side to the total area of the lead insulation layer 79 in a top view, the limiting current when oxygen was pumped from the surroundings of the reference electrode 42 to the surroundings of the outer pump electrode 23, the evaluation results of stabilization time, and the evaluation results of NOx detection accuracy are illustrated in Table 1.

TABLE 1

| | Porosity of | Ratio of densec region | Presence of | Limiting | Evaluation of | Evaluation Nox |
|---|---|---|---|---|---|---|
| | internal | to total area of | preliminary | current | stabilization | detection |
| Level | lead [%] | lead insulation layer [%] | chamber | [uA] | time | accuracy |
| Example 1 | 15 | 100 | No | 1.0 | A | A |
| Example 2 | 15 | 100 | No | 2.0 | A | A |
| Example 3 | 6 | 50 | No | 0.3 | B | B |
| Example 4 | 15 | 80 | No | 0.7 | A | A |
| Example 5 | 10 | 70 | No | 0.5 | B | A |
| Example 6 | 10 | 90 | No | 0.5 | A | A |
| Example 7 | 8 | 75 | No | 0.4 | B | B |
| Example 8 | 10 | 80 | No | 0.7 | A | A |
| Example 9 | 40 | 100 | No | 2.0 | A | A |
| Example 10 | 15 | 100 | Yes | 2.0 | A | A |
| Comparative | 5 | 100 | No | 0.2 | A | F |
| Example 1 | | | | | | |
| Comparative | 15 | 40 | No | 1.0 | F | A |
| Example 2 | | | | | | |

As shown in Table 1, in Examples 1 to 10, in which the porosity of the internal lead 78b is more than 5% and 40% or less, the ratio of the dense region, from the end on the reference electrode 42 side, to the total area of the lead insulation layer 79 in the top view is 50% or more, and the limit current when oxygen is pumped from the surroundings of the reference electrode 42 to the surroundings of the outer pump electrode 23 is 0.3 μA or more, the evaluation of stabilization time was “A” or “B”, and the evaluation of NOx detection accuracy was also “A” or “B”. In other words, examples 1 to 10 had a short stabilization time and good NOx detection accuracy. In contrast, in Comparative Example 1, the evaluation of NOx detection accuracy was “F”. In Comparative Example 1, the porosity of the internal lead 78b was 5%, indicating that the internal lead 78b was dense, and the limiting current when oxygen was pumped from the surroundings of the reference electrode 42 to the surroundings of the outer pump electrode 23 was 0.2 μA. It is believed that the oxygen concentration around the reference electrode 42 is difficult to maintain and the NOx detection accuracy is not good because the movement of the reference gas between the surroundings of the reference electrode 42 and the outside of the sensor element 101 is greatly restricted. In Comparative Example 2, the evaluation of stabilization time was “F”. In Comparative Example 2, the ratio of the dense region, from the end on the reference electrode 42 side, to the total area of the lead insulation layer 79 in the top view is 40%, indicating that the ratio of the dense region is low. As a result, the amount of water adsorbed by the lead insulation layer 79 during the non-operation period of the sensor element 101 is large, and it takes time for the water to drain out of the lead insulation layer 79, resulting in a longer stabilization time. Based on the comparisons, it is considered that in Examples 1 to 10, the porosity of the internal lead 78b is more than 5% and 40% or less, the ratio of the dense region from the end on the reference electrode 42 side to the total area of the lead insulation layer 79 in the top view is 50% or more, and the limiting current when oxygen is pumped from the surroundings of the reference electrode 42 to the surroundings of the outer pump electrode 23 is 0.3 μA or more. This configuration likely contributes to a shorter stabilization time while reducing the decrease in NOx detection concentration.

In Addition, in Examples 1 to 10, the evaluation of stabilization time was “A” for Examples 1, 2, 4, 6, and 8 to 10, in which the ratio of the dense region from the end on the reference electrode 42 side to the total area of the lead insulation layer 79 in the top view was 80% or more. In contrast, the evaluation of stabilization time was “B” for Examples 3, 5, and 7, in which this ratio was 50% or more but less than 80%. Therefore, it is considered preferable for the ratio of the dense region from the end on the reference electrode 42 side to the total area of the lead insulation layer 79 in the top view to be 80% or more.

Furthermore, in Examples 1 to 10, the evaluation of NOx detection accuracy was “A” for Examples 1, 2, 4 to 6, and 8 to 10, in which the limiting current when oxygen is pumped from the surroundings of the reference electrode 42 to the surroundings of the outer pump electrode 23 was 0.5 μA or more. In contrast, the evaluation of NOx detection accuracy was “B” for Examples 3 and 7, in which the limiting current was 0.3 μA or more but less than 0.5 μA. Therefore, it is considered preferable for the limiting current to be 0.5 μA or more.

What is claimed is:

1. A sensor element configured to detect a specific gas concentration in a measurement-object gas, the sensor element comprising:
an element body with an oxygen-ion-conductive solid electrolyte layer and a measurement-object gas flow section located inside the element body that introduces and flows the measurement-object gas;
a reference electrode disposed inside the element body;
a measurement-object gas side electrode disposed on a portion of the element body that is exposed to the measurement-object gas;
a first conducting section including a reference electrode terminal disposed on an outside of the element body, and a reference electrode lead that is electrically continuous between the reference electrode terminal and the reference electrode; and
a lead insulation layer covering a portion of the reference electrode lead that is located inside the element body,
wherein the reference electrode lead is configured to flow a reference gas, which serves as a reference for detecting the specific gas concentration in the measurement-object gas, to the reference electrode, and
a region of 50% or more, from an end on the reference electrode side, of the total area of the lead insulation layer in a planar view is configured as a dense region with a porosity of 5% or less.

2. The sensor element according to claim 1,
wherein the region of 80% or more, from the end on the reference electrode side, of the total area in the planar view is configured as the dense region.

3. The sensor element according to claim 1,
wherein the limiting current when oxygen is pumped from the surroundings of the reference electrode to the surroundings of the measurement-object gas side electrode is 0.3 μA or more.

4. The sensor element according to claim 3,
wherein the limiting current is 0.5 μA or more.

5. The sensor element according to claim 1,
wherein the porosity of a portion of the reference electrode lead that is located inside the element body is more than 5% and 40% or less.

6. The sensor element according to claim 1,
wherein a cross-sectional area and/or a porosity of a portion of the reference electrode lead that is located inside the element body is greater on the reference electrode side than on the reference electrode terminal side.

7. The sensor element according to claim 1, further comprising:
a heater disposed inside the element body and heating the element body;
a second conducting section including a heater terminal disposed on the outside of the element body, and a heater lead that is electrically continuous between the heater terminal and the heater; and
a heater insulation layer covering both the heater and a portion of the heater lead that is located inside the element body,
wherein the lead insulation layer and the heater insulation layer are independently provided such that they do not connect with each other.

8. The sensor element according to claim 1, further comprising:
a heater disposed inside the element body and heating the element body;
a second conducting section including a heater terminal disposed on the outside of the element body, and a heater lead that is electrically continuous between the heater terminal and the heater; and
a heater insulation layer covering both the heater and a portion of the heater lead that is located inside the element body,
wherein the element body includes a communication hole that connects the heater insulation layer and the lead insulation layer, and
a porosity of the heater insulation layer is 5% or less.

9. A gas sensor comprising:
the sensor element according to claim 1.
